(19) United States
(12) Patent Application Publication
(10) Pub. No.: US 2022/0293210 A1
(43) Pub. Date: Sep. 15, 2022

(54) SINGLE-CELL MODELING OF CLINICAL DATA TO DETERMINE RED BLOOD CELL REGULATION

(71) Applicant: The General Hospital Corporation, Boston, MA (US)

(72) Inventors: John M. Higgins, Cambridge, MA (US); Anwesha Chaudhury, Boston, MA (US)

(21) Appl. No.: 17/636,272

(22) PCT Filed: Aug. 20, 2020
(86) PCT No.: PCT/US2020/047173

§ 371 Date: Feb. 17, 2022

Related U.S. Application Data
(60) Provisional application No. 62/889,116, filed on Aug. 20, 2019.

(51) Int. Cl. G16B 5/20; A61K 33/26; G16H 50/20; G16H 10/40

(52) U.S. Cl. CPC G16B 5/20; A61K 33/26; G16H 50/20; G16H 10/40

(57) ABSTRACT

A method includes receiving data representing a first complete blood count (CBC) measured from a first sample of red blood cells (RBCs) from a subject and data representing a second CBC measured from a second sample of RBCs from the subject, each of the first and second CBCs including a volume and a hemoglobin content of each of the RBCs in the respective sample, the first and second samples being different samples corresponding to different times. Parameters representing RBC population dynamics for the subject are calculated based on the volume and the hemoglobin content for the RBCs in each of the first and second samples and a time between the first and second samples. A pathophysiological state of the subject is determined based on the one or more parameters representing the RBC population dynamics.

### CLAIM OF PRIORITY

This application claims the benefit of U.S. Provisional Patent Application No. 62/889,116, filed on Aug. 20, 2019. The entire contents of the foregoing is incorporated herein by reference.

### FEDERALLY SPONSORED RESEARCH OR DEVELOPMENT

This invention was made with Government support under 1DP2DK098087 awarded by the National Institutes of Health. The Government has certain rights in the invention.

### FIELD OF THE INVENTION

The invention relates to techniques for single-cell modeling of clinical data, such as data obtained from blood tests, to determine dynamics of red blood cell regulation.

### BACKGROUND

A complete blood count (CBC), a widely used clinical test, summarizes basic features of circulating red blood cell (RBC) populations. The CBC can include measurement of the variation in volume of individual red blood cells (RBCs). The systems controlling the number, size, hemoglobin concentrations, and other characteristics of circulating human RBCs measured by the CBC are poorly understood. After release from the bone marrow, RBCs undergo reduction in both volume and total hemoglobin content. In a healthy individual, after about 120 days, the RBCs are typically removed or cleared.

### SUMMARY

Low blood count is a fundamental disease state and is often an early sign of illnesses including infection, cancer, malnutrition, or combinations of them, among others. However, current understanding of the homeostatic response to blood loss is limited, in part due to coarse interpretation of blood measurements. The techniques described here present a novel, unsteady-state modeling approach of the dynamics of clinically available single-cell measurements of volume and hemoglobin content of red blood cell (RBC) populations in response to controlled blood loss in humans. By modeling volume and hemoglobin dynamics of RBC populations, increased production of new RBCs can be detected earlier than is currently detectable clinically, and a previously unrecognized decreased RBC turnover can be detected. The model provides a personalized dimensionless ratio that quantifies the balance between increased production and delayed clearance for each individual and may enable earlier detection of both blood loss and the homeostatic response that blood loss elicits.

In general, in a first aspect, a method includes receiving data representing a first complete blood count (CBC) measured from a first sample of red blood cells (RBCs) from a subject, the first CBC including a volume and a hemoglobin content of each of the RBCs in the first sample, receiving data representing a second CBC measured from a second sample of RBCs from the subject, the first and second samples being different samples corresponding to different times, the second CBC including a volume and a hemoglobin content of each of the RBCs in the second sample, calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate, and determining a pathophysiological state of the subject based on the one or more parameters representing the RBC population dynamics.

In general, in a second aspect, combinable with the first aspect, determining the pathophysiological state of the subject includes determining, based on the one or more parameters, at least one of a RBC clearance rate, a RBC production rate, or a RBC age distribution.

In general, in a third aspect, combinable with any of the first or second aspects, determining the pathophysiological state of the subject includes determining, based on the one or more parameters, at least one of a rate of change in white blood cell count, a rate of change in platelet count, a rate of blood loss, or a rate of bone marrow cellular output.

In general, in a fourth aspect, combinable with any of the first through third aspects, determining the pathophysiological state of the subject includes determining, based on the one or more parameters, information indicative of a degree of morbidity of the subject, the information including at least one of: information indicative of the presence of an infection, information indicative of the presence of malignancy, information indicative of the presence of anemia, or information indicative of the presence of diabetes.

In general, in a fifth aspect, combinable with any of the first through fourth aspects, the one or more parameters include at least one of a rate of RBC volume change or a variation of RBC volume change.

In general, in a sixth aspect, combinable with any of the first through fifth aspects, the one or more parameters include at least one of a rate of RBC hemoglobin reduction or a variation of RBC hemoglobin reduction.

In general, in a seventh aspect, combinable with any of the first through sixth aspects, calculating one or more parameters representing RBC population dynamics for the subject includes calculating a probability density of RBC volume, RBC hemoglobin content, and RBC age as a function of time.

In general, in an eighth aspect, combinable with any of the first through seventh aspects, the probability density as a function of time is determined according to the expression −∇·(Pf)+∇·(D∇P)+b(v, h)−d(v, h), where P is a RBC volume-hemoglobin probability distribution, f is a drift term, D is a diffusion matrix, b(v, h) is RBC production defined as a function of RBC volume and hemoglobin content, and d(v, h) is RBC clearance defined as a function of RBC volume and hemoglobin content.

In general, in a ninth aspect, combinable with any of the first through eighth aspects, the method includes calculating a RBC age distribution for the subject based on the one or more parameters and at least one of the first CBC or the second CBC.

In general, in a tenth aspect, combinable with any of the first through ninth aspects, the method includes receiving a hemoglobin A1c (HbA1c) measurement for the subject, determining a HbA1c level indicative of diabetes or prediabetes for the subject by adjusting a nominal HbA1c level based on the RBC age distribution, and administering treatment for diabetes or prediabetes to the subject in response to a determination that the HbA1c measurement for the subject exceeds the HbA1c level for the subject.

In general, in an eleventh aspect, combinable with any of the first through tenth aspects, the method includes receiving a HbA1c measurement for the subject, determining a HbA1c level indicative of diabetes or prediabetes for the subject by adjusting a nominal HbA1c level based on the RBC age distribution, and adjusting treatment for diabetes or prediabetes to the subject in response to a determination that the HbA1c measurement for the subject exceeds the HbA1c level for the subject.

In general, in a twelfth aspect, combinable with any of the first through eleventh aspects, the method includes administering a dose of iron supplementation to the subject in response to a determination that the change in the RBC clearance rate does not meet a predefined threshold.

In general, in a thirteenth aspect, combinable with any of the first through twelfth aspects, a system includes one or more processors and memory storing instructions which, when executed by the one or more processors, cause the one or more processors to: receive data representing a first CBC measured from a first sample of RBCs from a subject, the first CBC including a volume and a hemoglobin content of each of the RBCs in the first sample, receive data representing a second CBC measured from a second sample of RBCs from the subject, the first and second samples being different samples corresponding to different times, the second CBC including a volume and a hemoglobin content of each of the RBCs in the second sample, calculate one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate, and determine a pathophysiological state of the subject based on the one or more parameters representing the RBC population dynamics.

In general, in a fourteenth aspect, combinable with any of the first through thirteenth aspects, a non-transitory computer-readable storage medium storing instructions which, when executed by one or more processors, cause the one or more processors to perform operations including: receiving data representing a first CBC measured from a first sample of RBCs from a subject, the first CBC including a volume and a hemoglobin content of each of the RBCs in the first sample, receiving data representing a second CBC measured from a second sample of RBCs from the subject, the first and second samples being different samples corresponding to different times, the second CBC including a volume and a hemoglobin content of each of the RBCs in the second sample, calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate, and determining a pathophysiological state of the subject based on the one or more parameters representing the RBC population dynamics.

In general, in a fifteenth aspect, combinable with any of the first through fourteenth aspects, a method includes receiving data representing a first CBC measured from a first sample of RBCs from a subject, the first CBC including a volume and a hemoglobin content of each of the RBCs in the first sample, receiving data representing a second CBC measured from a second sample of RBCs from the subject, the first and second samples being different samples corresponding to different times, the second CBC including a volume and a hemoglobin content of each of the RBCs in the second sample, calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate, and administering treatment to the subject for a morbidity in response to a determination that at least one of the one or more parameters does not meet a predefined threshold.

In general, in a sixteenth aspect, combinable with any of the first through fifteenth aspects, the morbidity includes at least one of an infection, a malignancy, anemia, or diabetes.

In general, in a seventeenth aspect, combinable with any of the first through sixteenth aspects, the one or more parameters include at least one of a rate of RBC volume change or a variation of RBC volume change.

In general, in an eighteenth aspect, combinable with any of the first through seventeenth aspects, the one or more parameters include at least one of a rate of RBC hemoglobin reduction or a variation of RBC hemoglobin reduction.

In general, in a nineteenth aspect, combinable with any of the first through eighteenth aspects, calculating one or more parameters representing RBC population dynamics for the subject includes calculating a probability density of RBC volume, RBC hemoglobin content, and RBC age as a function of time.

In general, in a twentieth aspect, combinable with any of the first through nineteenth aspects, the probability density as a function of time is determined according to the equation −∇·(Pf)+∇·(D∇P)+b(v, h)−d(v, h), where P is a RBC volume-hemoglobin probability distribution, f is a drift term, D is a diffusion matrix, b(v, h) is RBC production defined as a function of RBC volume and hemoglobin content, and d(v, h) is RBC clearance defined as a function of RBC volume and hemoglobin content.

In general, in a twenty-first aspect, combinable with any of the first through twentieth aspects, the method includes calculating a RBC age distribution for the subject based on the one or more parameters and at least one of the first CBC or the second CBC.

In general, in a twenty-second aspect, combinable with any of the first through twenty-first aspects, the method includes receiving a HbA1c measurement for the subject, determining a HbA1c level indicative of diabetes or prediabetes for the subject by adjusting a nominal HbA1c level based on the RBC age distribution, and administering treatment for diabetes or prediabetes to the subject in response to a determination that the HbA1c measurement for the subject exceeds the HbA1c level for the subject.

In general, in a twenty-third aspect, combinable with any of the first through twenty-second aspects, a method includes receiving a HbA1c measurement for the subject, determining a HbA1c level indicative of diabetes or prediabetes for the subject by adjusting a nominal HbA1c level based on the RBC age distribution, and adjusting treatment for diabetes or prediabetes to the subject in response to a determination that the HbA1c measurement for the subject exceeds the HbA1c level for the subject.

In general, in a twenty-fourth aspect, combinable with any of the first through twenty-third aspects, the method includes administering a dose of iron supplementation to the subject in response to a determination that the change in the RBC clearance rate does not meet a predefined threshold.

In general, in a twenty-fifth aspect, combinable with any of the first through twenty-fourth aspects, a system includes one or more processors, and memory storing instructions which, when executed by the one or more processors, cause the one or more processors to: receive data representing a first CBC measured from a first sample of RBCs from a subject, the first CBC including a volume and a hemoglobin content of each of the RBCs in the first sample, receive data representing a second CBC measured from a second sample of RBCs from the subject, the first and second samples being different samples corresponding to different times, the second CBC including a volume and a hemoglobin content of each of the RBCs in the second sample, calculate one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate, and indicate treatment for the subject for a morbidity in response to a determination that at least one of the one or more parameters does not meet a predefined threshold.

In general, in a twenty-sixth aspect, combinable with any of the first through twenty-fifth aspects, a non-transitory computer-readable storage medium storing instructions which, when executed by one or more processors, cause the one or more processors to perform operations comprising: receiving data representing a first CBC measured from a first sample of RBCs from a subject, the first CBC including a volume and a hemoglobin content of each of the RBCs in the first sample, receiving data representing a second CBC measured from a second sample of RBCs from the subject, the first and second samples being different samples corresponding to different times, the second CBC including a volume and a hemoglobin content of each of the RBCs in the second sample, calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate, and indicating treatment for the subject for a morbidity in response to a determination that at least one of the one or more parameters does not meet a predefined threshold.

One or more of the above aspects may provide the following advantages. In general, the techniques described here use single-cell measurements made available through routine clinical tests, along with knowledge of human pathophysiologic functions, to infer features of an individual's pathophysiologic state that are not feasible or possible to measure directly. These newly quantifiable features of an individual's pathophysiologic state enable better understanding of the individual's homeostatic response to, for example, blood loss and are useful, for example, in making diagnoses or monitoring and personalizing treatment. Some of these diagnoses and treatment decisions are not possible to make at all without the information provided by the techniques described here. Other diagnoses and treatment decisions can currently be made, but the information provided by using the disclosed techniques potentially allows such diagnoses and treatment decisions to be made earlier or more accurately, or both, thereby potentially allowing a healthcare professional to administer care to the subject before the onset of the condition or the manifestation of uncomfortable or progressive or advanced symptoms of the condition

Unlike other methods which rely on sophisticated or resource-intensive techniques to indirectly infer some RBC dynamics, the techniques described here use single-cell measurements of RBC volume and hemoglobin mass made available through routine clinical tests to quantify RBC dynamics. This allows the disclosed techniques to be easily applied with high throughput, creating a shorter path to clinical translation of any potential insights. In addition, by defining RBC dynamics as a function of time and incorporating two or more distinct measurements (e.g., two or more distinct blood counts), the techniques describe here are able to detect more subtle changes than steady or quasi-steady state systems or systems that rely on only a single blood count.

Unless otherwise defined, all technical and scientific terms used herein have the same meaning as commonly understood by one of ordinary skill in the art. Methods and materials described herein are for illustration purposes; other suitable methods and materials known in the art can also be used. The materials, methods, and examples are and not intended to be limiting. All publications, patent applications, patents, sequences, database entries, and other references mentioned herein are incorporated by reference in their entirety. References parenthetically cited are listed herein below. In case of conflict, the present specification, including definitions, will control.

Other features and advantages will be apparent from the following detailed description and figures, and from the claims.

### DESCRIPTION OF DRAWINGS

FIG. 1 is a schematic diagram of an example of a static red blood cell (RBC) population volume and hemoglobin distribution.

FIG. 2 is a schematic diagram of an example of a model of single-RBC volume-hemoglobin dynamics.

FIGS. 3A and 3B are box plots of RBC population and RBC dynamics for subjects before and after blood loss.

FIGS. 4A to 4F illustrate simulations of an example model of population dynamics of a population of RBCs.

FIGS. 5A to 5G is a schematic diagram of various features of an example model of population dynamics of a population of RBCs.

FIGS. 6A to 6D illustrates box plots of mean corpuscular hemoglobin concentration (MCHC) and coefficient of variation in single-RBC hemoglobin concentration (CHDW) for subjects following blood loss.

FIG. 7 is a schematic diagram of an example of modeling integrated serial complete blood count (CBCs) measurements into the parameter estimation process.

FIG. 8 is an example process for determining a pathophysiologic state of a subject.

FIG. 9 is a block diagram of an example computing system.

### DETAILED DESCRIPTION

The typical adult human produces about two million red blood cells (RBCs) per second, with a similar rate of clearance of old RBCs after they have circulated for about 90 to 120 days. RBC lifespan is tightly controlled within each person but can vary from one person to the next (see, e.g., Cohen et al., 2008; Malka et al., 2014). The volume of a typical RBC decreases by about 30% (e.g., from about 115 fl to about 80 fl) over the course of the RBC's lifespan. Similarly, the hemoglobin mass of a typical RBC decreases by about 20% (e.g., from about 35 pg to 28 pg) over the course of the RBC's lifespan, with the average hemoglobin concentration increasing modestly over that same time frame (see, e.g., Malka et al., 2014; Willekens et al., 2008).

The circulating population of RBCs in an individual is thus continuously changing through a dynamic process that includes production (erythropoiesis), maturation and aging, and clearance (see, e.g., Bunn, 2013). In healthy individuals, characteristics (e.g., RBC volume and hemoglobin content) of the RBC population can remain stable during this dynamic process. However, these RBC population dynamics may change due to mechanisms leading to or associated with a pathological condition (e.g., a disease, an infection, a malignancy, or combinations of them, among others). For example, for a particular cellular characteristic, such as volume or hemoglobin content, that changes over the course of a RBC's lifespan, the rate of this change across the population of RBCs from an individual with some disease can be different from the rate of this change across the population of RBCs from the same or different individual without the disease.

Certain clinical tests can provide insight into an individual's RBC population at a given time. For example, routine complete blood counts (CBCs) include measurements of single-cell volume and hemoglobin for a large number (e.g., about 50,000) of individual RBCs in a blood sample from an individual, effectively providing a snapshot of the current RBC population for the individual. In addition, some of the youngest RBCs (sometimes referred to as “reticulocytes”) can be identified in these counts because they generally include ribonucleic acid (RNA) remnants in their membranes (see, e.g., d'Onofrio et al., 1995). However, these tests, on their own, cannot measure certain features of an individual's pathophysiologic state, such as RBC clearance or production rate or variance in RBC clearance or production rate, with the necessary accuracy or at all.

The techniques described here use the single-cell measurements made available through routine clinical tests, along with knowledge of human pathophysiologic functions, to infer features of an individual's pathophysiologic state that are not feasible or possible to measure directly. Specifically, the described technology uses knowledge of functions such as RBC maturation and volume-hemoglobin dynamics to derive a model that relates the probability density of RBC population volume, hemoglobin mass, and age as a function of time. The model has a level of abstraction coinciding with that of measurements made by routine clinical tests (e.g., complete blood and reticulocyte counts) such that the variables in the model represent quantities that are measured by the routine clinical tests. The model contains parameters that describe, for example, rates and variances of pathophysiologic functions that either cannot be measured directly or require highly sophisticated methods or levels of resources that make their use in routine settings infeasible.

These newly quantifiable features of an individual's pathophysiologic state are useful in making diagnoses or monitoring and personalizing treatment as detailed herein. Some of these diagnoses and treatment decisions are not possible to make at all without the information provided by the model. Other diagnoses and treatment decisions can currently be made, but the information provided by the model allows them to be made earlier or more accurately, or both.

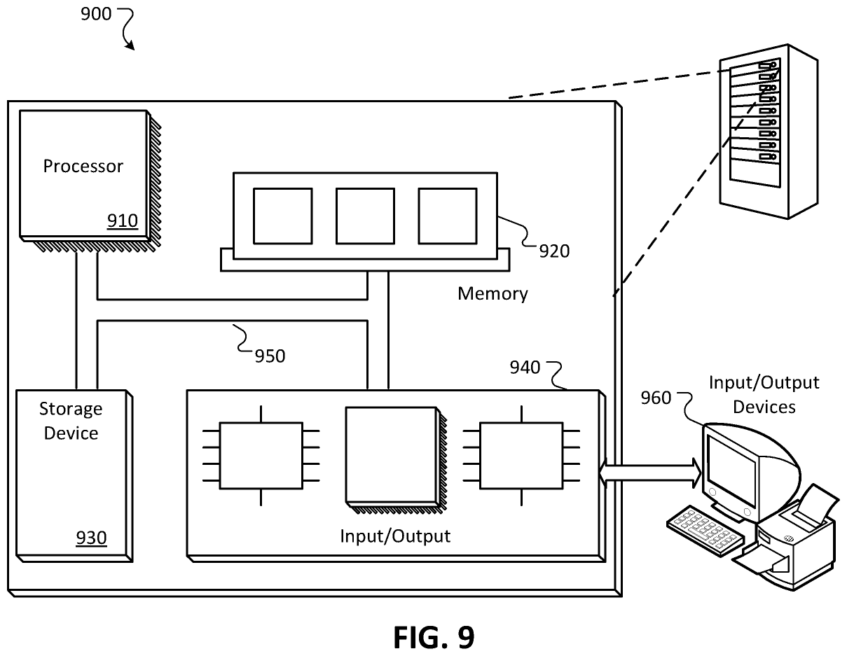

The techniques described here can be implemented using various hardware and software components, as described below. In some examples, the techniques described here are implemented using a computing system, such as the computing system 900 shown in FIG. 9. The technology can be deployed in a distributed or centralized fashion, and can be implemented as a standalone module (e.g., using a computing system at a healthcare facility) for providing results to patients and healthcare professionals to inform treatment or diagnosis, or integrated with another system or device such as an electronic medical record system or hematology analyzer, among others.

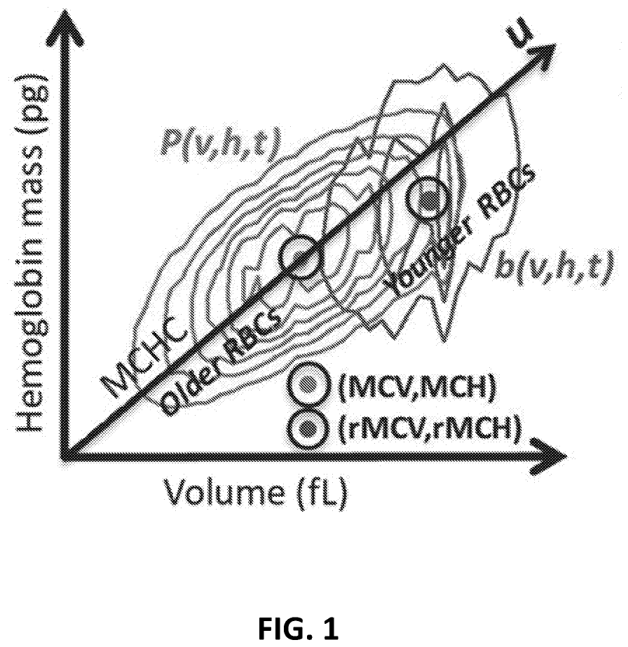

Referring to FIG. 1, a schematic diagram of a static RBC population volume and hemoglobin distribution is shown. In this example, a routine CBC samples the single-cell volume and hemoglobin for about 50,000 individual RBCs in a sample, although greater or fewer RBCs can be sampled in various implementations. These measurements can be used to produce a two-dimensional single-RBC volume-hemoglobin distribution (P(v, h, t)) for the sample representative of all RBCs in an individual's circulation at a particular time. In this example, the variable v represents the volume of a RBC in the sample, h represents the hemoglobin mass of the RBC in the sample, and t represents the time of the sample. Reticulocytes can also be identified in these counts (e.g., through detection of RNA remnants in their membranes as described in, e.g., d'Onofrio et al., 1995), and a distribution (b(v, h, t)) of these young RBCs at time t can be determined. The line (u) extending from the origin shows the mean corpuscular hemoglobin concentration (MCHC) for the sampled population. The typical healthy RBC follows a volume-hemoglobin (v, h) trajectory along the line (u) as it ages until it is eventually cleared in the lower left (low u). This major axis of the distribution along the line u provides a rough estimate of RBC age, with a point of the distribution appearing higher along the line u corresponding to a RBC having a younger age.

In some examples, static averages of marginal volume and hemoglobin distributions and other bulk blood characteristics are determined. These characteristics include MCHC, hemoglobin concentration per unit volume blood (HGB), hematocrit (HCT, volume fraction of RBCs), mean corpuscular volume (MCV), reticulocyte mean corpuscular volume (rMCV), mean corpuscular hemoglobin mass (MCH), reticulocyte mean corpuscular hemoglobin mass (rMCH), and the coefficient of variation in RBC volume (red cell distribution width or RDW), among others. For example, FIG. 1 plots the MCV, MCH within the contours of the RBC population volume-hemoglobin distribution (P(v, h, t)) and rMCV, rMCH within the contours of the reticulocyte volume-hemoglobin distribution (b(v, h, t)).

In some implementations, the single-cell measurements in each routine CBC can be directly used to inform clinical care. For example, anemia, a condition defined by a reduction of HGB or HCT (or both), is often the first sign of many major diseases including cancer, infection, heart failure, autoimmune disease, and malnutrition, among others. Thus, understanding the single-cell dynamics of the homeostatic response to blood loss can provide insight into the development and progression of many diseases and enhance our ability to diagnose, monitor, and intervene most effectively.

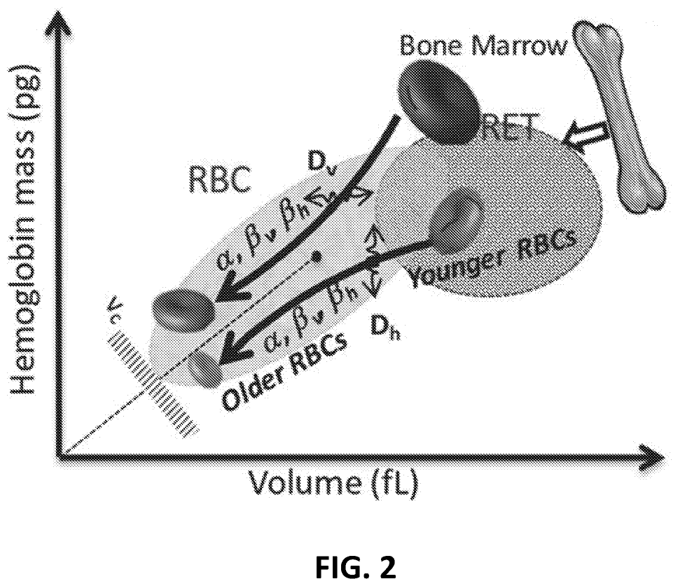

FIG. 2 shows a schematic diagram of a model of single-RBC volume-hemoglobin dynamics in accordance with an aspect of the present disclosure. As described above, the composition of the circulating RBC population is determined by a dynamic process that includes production, maturation and aging over the RBC lifespan, and clearance. During production (erythropoiesis), reticulocytes (RET) develop in the bone marrow and then enter circulation where they mature into RBCs, as shown in the top right portion of FIG. 2. As each RBC ages, it loses about 30% of its volume and about 20% of its hemoglobin during its 90-120 day lifespan. As the single-RBC volume and hemoglobin continue to fall, the probability of clearance increases dramatically as the RBC's trajectory approaches the clearance boundary region v_{c}.

The RBC population volume-hemoglobin distribution (P(v, h, t)) is therefore determined by a time-dependent production distribution (b(v, h, t)), dynamics, and a clearance distribution (d (v, h, t)) (e.g., a volume-hemoglobin distribution of RBCs cleared from circulation at a time t). Each routine CBC with a reticulocyte count provides an estimate of both P(v, h, t) and b(v, h, t). The dynamics of P(v, h) can be modeled as a drift-diffusion process (∇(N)+∇(D∇P)), and the functional specification of the drift, diffusion, and clearance terms can be guided by knowledge of in vivo RBC volume and hemoglobin dynamics. In some implementations, this can be done, for example, using techniques described in the following publications: Bosman et al. 2008. Erythrocyte ageing in vivo and in vitro: structural aspects and implications for transfusion. Transfus Med 18:335-347. doi:10.1111/j.1365-3148.2008.00892; Franco R S. 2008. The measurement and importance of red cell survival. Am J Hematol 84:109-114. doi:10.1002/ajh.21298; Gifford et al. 2006. A detailed study of time-dependent changes in human red blood cells: from reticulocyte maturation to erythrocyte senescence. Br J Haematol 135:395-404. doi:10.1111/j.1365-2141.2006.06279.x; Waugh et al. 1992. Rheologic properties of senescent erythrocytes: loss of surface area and volume with red blood cell age. Blood 79:1351 LP-1358; and Willekens et al. 2008. Erythrocyte vesiculation: a self-protective mechanism? Br J Haematol 141:549-556. doi:10.1111/j.1365-2141.2008.07055.x, the entire contents of each of which are incorporated herein by reference.

Modeling the RBC population in this way has several advantages, including: (1) the (v, h) space is densely sampled during each routine CBC, (2) b(v, h, t) can be directly sampled with each CBC, (3) physiologic knowledge of the dynamics of (v, h) can guide the functional form of dP/dt (see, e.g., Lew et al. 1995. Generation of normal human red cell volume, hemoglobin content, and membrane area distributions by “birth” or regulation? Blood 86:334 LP-341; Waugh et al. 1992. Rheologic properties of senescent erythrocytes: loss of surface area and volume with red blood cell age. Blood 79:1351 LP-1358; and Higgins et al. 2010. Physiological and pathological population dynamics of circulating human red blood cells. Proc Natl Acad Sci 107:20587-20592. doi:10.1073/pnas.1012747107, the entire contents of each of which are incorporated herein by reference), (4) P(v, h, t) and b(v, h, t) can be repeatedly sampled more frequently (e.g., minutes) than the characteristic timescale in the system (e.g., the about 100-day RBC lifespan), and (5) inferred single-cell trajectories can easily be combined with electronic medical record data to understand phenotypic effects of dynamics and feedback.

Leveraging knowledge of human pathophysiologic functions, RBC population dynamics are described with a semi-mechanistic, non-steady state model that relates the probability density of RBC volume, hemoglobin mass, and/or age as a function of time

$$( e . g . , \frac{d}{dt} P ( v , h , a , t ) = f ( v , h , t ) ) .$$

Specifically, the RBC population dynamics are described according to Equation (1):

$$\frac{\partial P}{\partial t} = - ∇ \cdot ( Pf ) + ∇ \cdot ( D ∇ P ) + b ( v , h ) - d ( v , h ) \qquad ( 1 )$$

where −∇·(Pf) represents drift, ∇·(D∇P) represents diffusion, b(v, h, t) is a production term, and d(v, h, t) is a clearance term.

Analysis under the assumption of steady state shows that the drift term can be approximated as a function of the RBC's current (v, h) with an early fast phase of volume and hemoglobin reduction during which the hemoglobin concentration of young RBCs approaches the population mean (see, e.g., Higgins and Mahadevan, 2010). This fast phase is parameterized by Bᵥ and Bₕ and is followed by a slower phase of coordinated volume and hemoglobin reduction parameterized by α. Thus, in the Fokker-Planck equation describing the RBC population dynamics (Equation 1), the drift term is expressed as a combination of an initial fast phase, followed by a slow phase as:

$$f = \{ \begin{matrix}α {e}^{{β}_{v} ( v - h )} \\ α {e}^{{β}_{h} ( h - v )}\end{matrix} \qquad ( 2 )$$

The diffusive term

$$[ \begin{matrix}{D}_{v} & 0 \\ 0 & {D}_{h}\end{matrix} ]$$

is assumed constant without interaction and encapsulates the variation in the rates of volume and hemoglobin change (quantified by Dᵥ and Dₕ, respectively) from one RBC to the next and for the same RBC over time. The clearance term is approximated as a function of the RBC's current (v, h) and a parameter (v_{c}) for a clearance boundary region (see, e.g., Higgins and Mahadevan, 2010; Patel et al., 2015). Specifically, the clearance term is defined as follows:

$$d ( v , h ) = \frac{1}{1 + {e}^{Δ ( v , h )}}$$

$$Δ ( v , h ) = 100 \frac{cos ( θ ) \sqrt{{( v \overline{v} )}^{2} + {( h \overline{h} )}^{2}} - {v}_{c} \sqrt{{\overline{v}}^{2} + {\overline{h}}^{2}}}{{v}_{c} \sqrt{{\overline{v}}^{2} + {\overline{h}}^{2}}}$$

$$θ = {tan}^{- 1} ( \frac{\overline{h}}{\overline{v}} ) - {tan}^{- 1} ( \frac{h \overline{h}}{v \overline{v}} )$$

Here, v̅ and h̅ are the MCV and MCH, respectively, and v_{c} parameterizes the clearance boundary region (see, e.g., Higgins and Mahadevan, 2010; Patel et al., 2015).

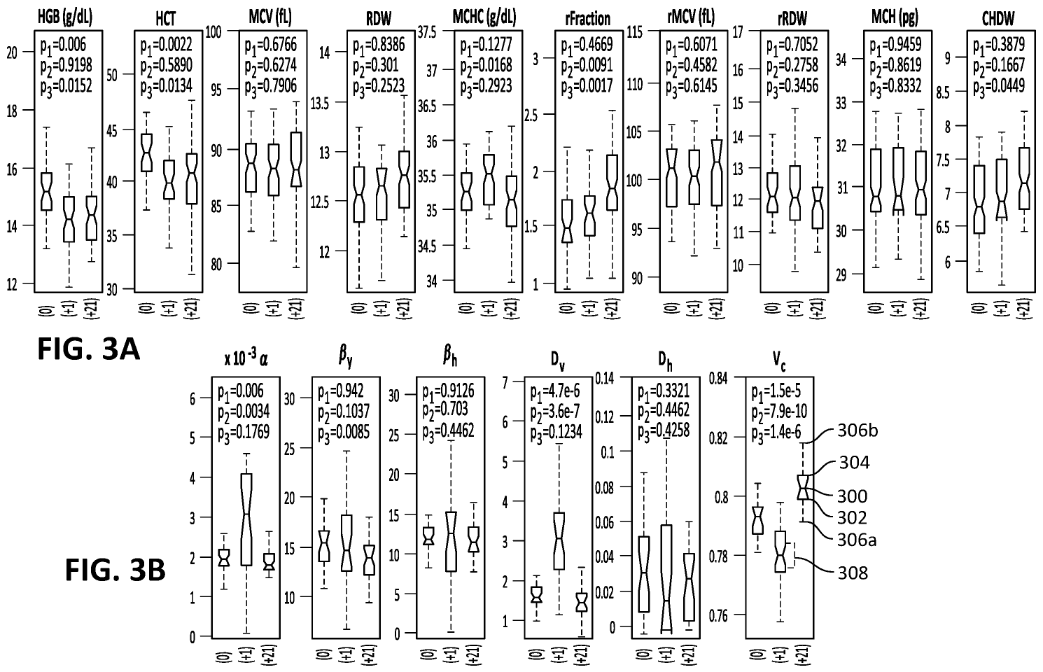

The effect of blood loss on transient RBC population dynamics was studied by collecting one unit of blood (corresponding to about a 10% blood loss) from each subject and estimating model parameters before and after. Statistics for the RBC population and RBC dynamics of this study are shown in FIGS. 3A and 3B. In particular, FIG. 3A shows the RBC population statistics obtained from CBCs for 28 healthy subjects before blood loss (depicted in the x axes of the figures as 0), 1-3 days after blood loss (depicted in the x axes of the figures as +1), and 21 days after blood loss (depicted in the x axes of the figures as +21), including HGB, HCT, MCV, RDW, MCHC, rFraction (percentage of identified reticulocytes), rMCV, rRDW (coefficient of variation in reticulocyte volume), MCH, and CHDW (coefficient of variation in single-RBC hemoglobin concentration). FIG. 3B shows single-RBC volume and hemoglobin dynamics statistics for the same 28 healthy subjects before blood loss (depicted in the x axes of the figures as 0), 1-3 days after blood loss (depicted in the x axes of the figures as +1), and 21 days after blood loss (depicted in the x axes of the figures as +21), including α, Bᵥ, Bₕ, Dᵥ, Dₕ, and v_{c}. In each of FIGS. 3A and 3B, box plots show the median (middle horizontal line 300), the 25^{th} (bottom horizontal line 302) and 75^{th} (top horizontal line 304) percentiles, and whiskers (306a, 306b) extend to data points not more than 1.5-times the interquartile range from the median. Notches 308 show a 95% confidence interval for the median, and any additional outliers are shown as discrete points. p₁ compares +0 with +1, p₂ compares +1 and +21, p₃ compares 0 and +21.

As shown in FIGS. 3A and 3B, significant blood loss triggers a rapid acellular fluid shift to restore intravascular volume. Intensive quantities, including HGB and HCT, change significantly immediately following blood loss due to this fluid shift, but single-RBC population statistics do not change significantly (FIG. 3A). By 21 days after blood loss, the CHDW and rFraction have increased significantly relative to the baseline. MCHC at 21 days has decreased relative to 1-3 days. On the other hand, the RBC dynamics are generally more sensitive to blood loss than RBC population statistics (FIG. 3B). Over the first 1-3 days following blood loss, the single-cell (v, h) dynamics for most subjects showed significant increases in model parameters α and Dᵥ and a decrease in v_{c} (FIG. 3B). Greater a reflects a faster reduction in (v, h) for the typical RBC or a longer RBC lifespan (since a is normalized by a nominal lifespan) or both. Greater Dᵥ reflects increased variation in the rate of RBC volume reduction, or a longer RBC lifespan, or both. Smaller v_{c} reflects delayed clearance of RBCs with (v, h) low enough to have been cleared prior to blood loss.

Note that RBCs are assumed to be lost in a volume- and hemoglobin-independent fashion, meaning that P(v, h, t) is not directly altered. This assumption is based on labeling studies which model the residual lifespan of labeled RBCs (after reinfusion and recollection) to infer that a blood draw is a random sample of RBCs of all ages (see, e.g., Franco, 2008; Franco et al., 2013; Khera et al., 2013a; Shrestha et al., 2016). The evidence for this assumption is indirect, relying on models of RBC lifespan distributions, and definitive establishment of its validity awaits the development of an accepted direct measurement or marker of RBC age. An individual can compensate for blood loss by increasing the rate of RBC production or by reducing the rate of clearance, or both. Production and clearance have baseline rates of ˜1% per day (see, e.g., Dornhorst, 1951; Franco et al., 2013). Under physiologic conditions, only the oldest RBCs are cleared (see, e.g., Cohen et al., 2008; Franco, 2008; Franco et al., 2013; Khera et al., 2013b). The gold standard “reticulocyte count” does not reliably detect increased production for about 5 days (see, e.g., Jelkmann and Lundby, 2011; Piva et al., 2015; Sieff, 2017), but the true production rate may increase earlier (e.g., through analysis of RBC dynamics).

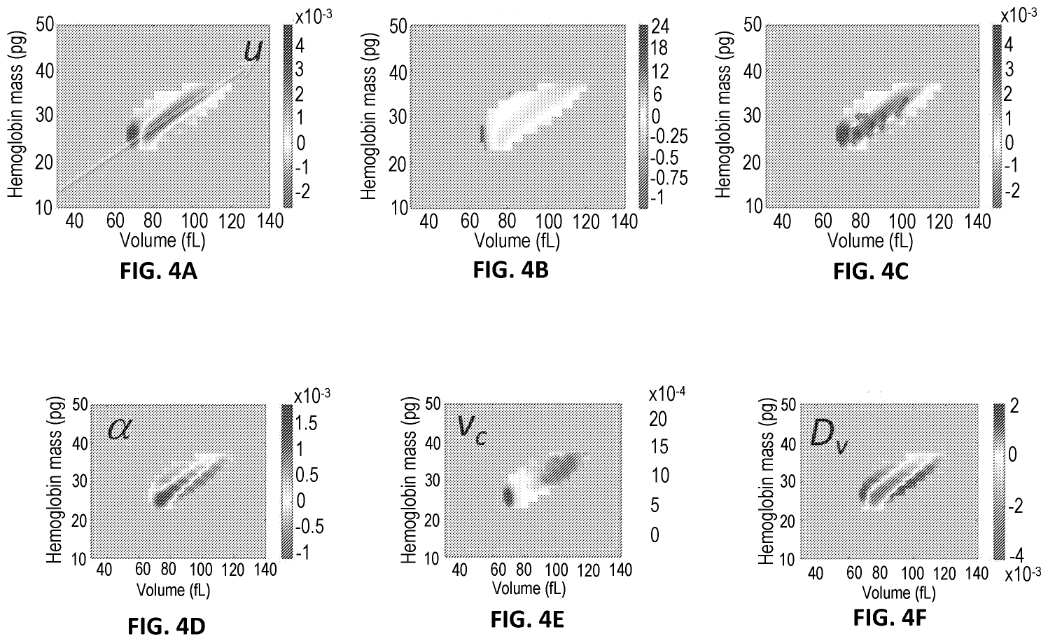

FIGS. 4A-4F show simulations of the model which identify regions of P(v, h) where the blood loss response causes the largest changes. In particular, FIGS. 4A and 4B show the absolute and relative changes, respectively, in the simulated single RBC volume-hemoglobin probability density (e.g., when setting Dᵥ′=4Dᵥ, α′=2α, and v_{c}′=0.9v_{c} to match the median changes shown in FIG. 3B). FIG. 4C shows the absolute changes of FIG. 4A with arrows depicting the typical movements in probability density 1-3 days after blood loss. FIGS. 4D, 4E, and 4F show the effects of isolated changes to individual parameters (α, v_{c}, and Dᵥ, respectively), with changes to α and v corresponding to retention of older RBCs (delayed clearance), and changes to Dᵥ adding density in the high-volume, low hemoglobin region where new RBCs appear, corresponding, in part, to increased production

In general, the simulations in FIGS. 4A-4F show an increase in the low-u region containing older cells, milder increase in the high-u, low-hemoglobin region containing young RBCs, and a balancing decrease along the u axis above the low tail. In other words, blood loss causes a shift of probability density from the central axis of the (v, h) distribution, mostly to the low volume-low hemoglobin tail. The empirical effect of blood loss response on the older cell fraction can be quantified by integrating P(u) one standard deviation below the median and lower. FIG. 4D shows a significant increase in the fraction of older RBCs for most subjects during the first 1-3 days after blood loss, consistent with a delayed clearance.

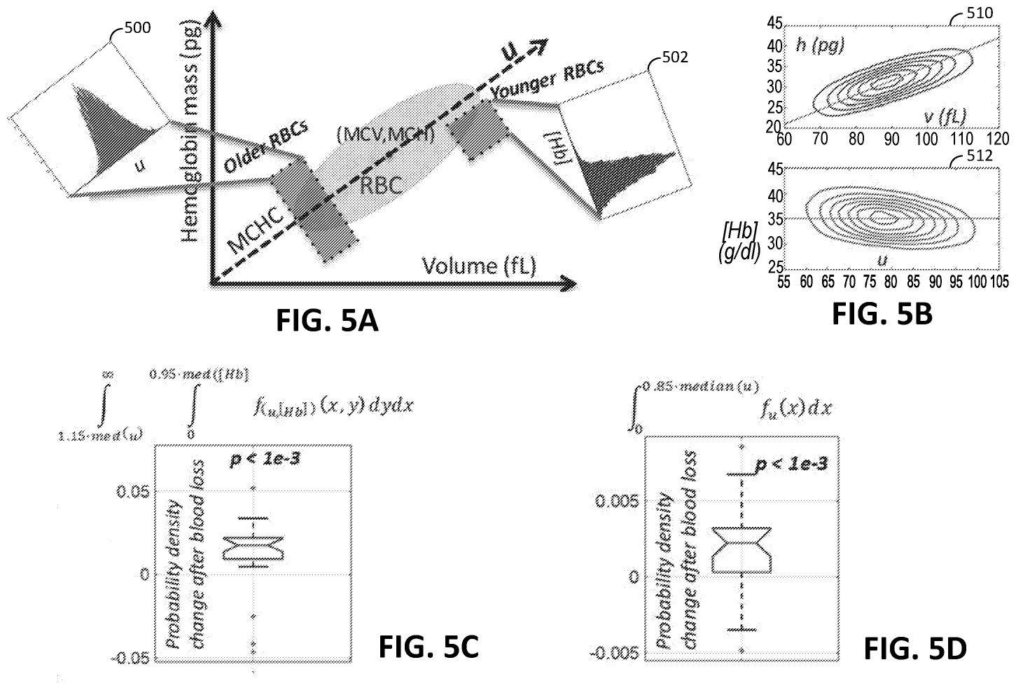

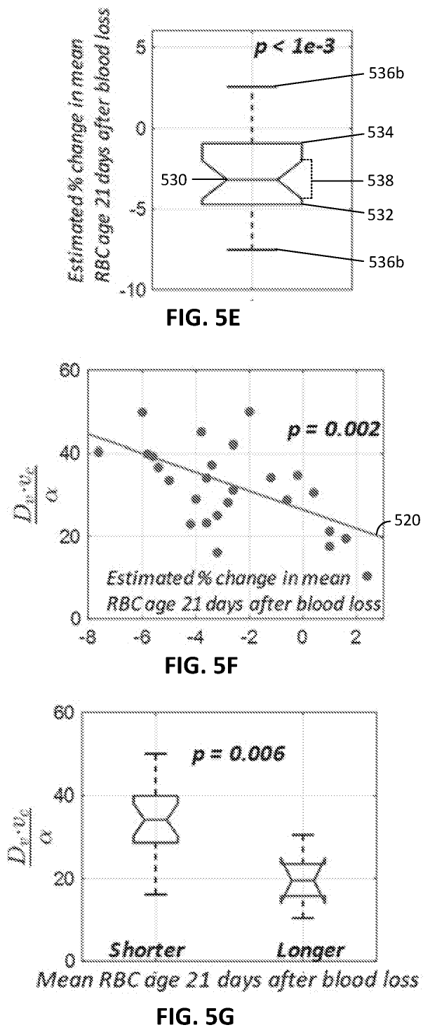

FIGS. 5A-5G schematically illustrate the mechanistic link provided by the single-cell model between dynamics of the (v, h) distribution and the balance between increased RBC production and delayed RBC clearance in response to blood loss. In particular FIG. 5A shows a schematic of the single-cell volume-hemoglobin distribution for RBCs. The major axis of the distribution (u) corresponds to the mean single-RBC hemoglobin concentration (MCHC). A RBC's position when projected onto u corresponds roughly to its age, with younger RBCs generally appearing in the upper right, and aging along the u axis toward the origin in the bottom left. Changes in the fraction of older RBCs can be compared by integrating density along u as shown in an inset 500. Changes in the fraction of newly produced RBCs can be compared by conditioning on higher u and integrating density along the hemoglobin axis as shown in an inset 502.

A graph 510 in FIG. 5B shows a typical (v, h) distribution for a subject (e.g., as produced from CBC measurements). A graph 512 shows the (v, h) distribution for the subject transformed onto the u-hemoglobin plane. As shown in FIG. 5C, the typical blood loss response after 1-3 days includes an increase in the fraction of newly produced cells which will have hemoglobin more than one standard deviation below the median and u more than one standard deviation above the median (p<1e-3), corresponding to the inset 502 in FIG. 5A and consistent with increased production. FIG. 5D shows that, 1-3 days following blood loss, the typical response also involves an increase in the fraction of older RBCs, located more than one standard deviation (e.g., about 15%) below the median u (p<1e-3), corresponding to the inset 500 and consistent with a delayed clearance.

As shown in FIG. 5E, the mean RBC age (M_{RBC}), as estimated by the glycated hemoglobin fraction, has decreased on average by about 4% after 21 days, but there is significant variation, with some subjects seeing an increase in M_{RBC}. In some examples, the model characterizes the relative balance between increased production and delayed clearance in each subject's blood response by the dimensionless parameter ratio

$$\frac{{D}_{v} \cdot {v}_{c}}{α} ,$$

as shown in FIG. 5F. The time-weighted average of this ratio after blood loss for each subject is significantly correlated with the estimated change in M_{RBC} (ρ=−0.59), suggesting that the model of (v, h) dynamics has accurately captured the

$$\frac{production}{clearance}$$

balance of the typical subject's blood loss response. The regression line 520 in FIG. 5F is a least-squares linear fit. Lastly, panel (G) of FIG. 5 shows that the dimensionless parameter ratio distinguishes subjects whose M_{RBC} becomes shorter (production-dominated) during response to blood loss from those whose M_{RBC} becomes longer (clearance-dominated). Note that the box plots in FIGS. 5C-5G show the median (middle horizontal line 530), the 25th (bottom horizontal line 532) and 75th (top horizontal line 534) percentiles, and whiskers (536a, 536b) extend to data points not more than 1.5-times the interquartile range from the median. Notches 538 show a 95% confidence interval for the median, and any additional outliers are shown as discrete points.

Newly produced RBCs have higher volume and lower hemoglobin concentration (see, e.g., d'Onofrio et al., 1995) and appear in the upper right of the (v, h) plane, or the bottom right quadrant of the u-h plane (see, e.g., panels (A) and (B) of FIG. 5). The simulations in FIGS. 4A-4F show that a simulated increase in Dᵥ is associated with an increase in P(v, h) in this region. Empirical evidence of increased production can be found by conditioning on u being more than one standard deviation above the median and then integrating the marginal hemoglobin distribution falling at least one standard deviation (e.g., about 5%) below the median. Panel (C) of FIG. 5 shows a significant increase for the typical subject, consistent with RBC production increasing days earlier than the current gold standard reticulocyte count (FIG. 3A). Application of the model in this study did not find any statistically significant sex-specific differences.

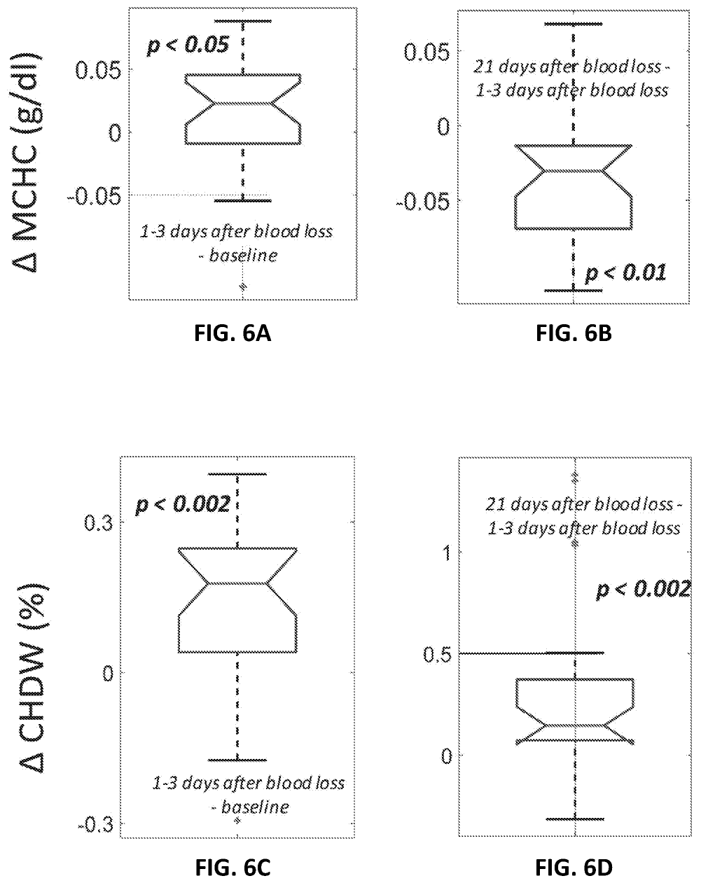

FIGS. 6A-6D show box plots of the MCHC and CHDW for subjects following blood loss. These plots show that the MCHC rise and fall and the sustained CHDW rise are consistent with a combination of delayed RBC clearance and increased RBC production. Single-RBC hemoglobin concentration ([Hb]) increases during the first few weeks of an RBC's lifespan and is then stable (see, e.g., Franco et al., 2013). Clearance delay would therefore enrich the fraction of older RBCs which have [Hb] slightly higher than the population mean at the expense of younger RBCs with relatively lower [Hb], and the population mean [Hb] (MCHC) would increase. On the other hand, increased production in isolation would reduce MCHC by adding more young RBCs with lower [Hb]. For the typical subject, it is found that MCHC increases shortly (e.g., 1-3 days) after blood loss as shown in FIG. 6A. The MCHC for the typical subject then falls, dropping below the baseline level by 21 days as shown in FIG. 6B. Both delayed clearance and increased production would be expected to increase the coefficient of variation in [Hb], CHDW, by enriching for RBCs with extreme [Hb], also consistent with measurements of CHDW, which increases shortly (e.g., 1-3 days) after blood loss (shown in FIG. 6C) and remains elevated relative to baseline even 21 days later (as shown in FIG. 6D).

The model described here enables estimation of the relative magnitudes of the production increase and clearance delay for individual subjects. The model thus suggests that the response to blood loss includes both delayed clearance (modeled as a higher α and lower v_{c}, or simply higher

$$\frac{α}{{v}_{c}} )$$

and increased production (modeled as a higher Dᵥ). These two component responses have opposite effects on the mean RBC age (M_{RBC}), with increased production enriching for younger RBCs and shortening M_{RBC}, and delayed clearance enriching for older RBCs and lengthening M_{RBC} M_{RBC} can be estimated in these nondiabetic subjects by measuring the glycated hemoglobin fraction. FIG. 5 shows that this estimated M_{RBC} has decreased by about 4% for the typical subject by 21 days, consistent with relatively higher increased production than delayed clearance for the typical subject, but the balance varies across subjects.

In some examples, the model is used to estimate the

$$\frac{production}{clearance}$$

response ratio for each subject as a dimensionless number:

$$\frac{{D}_{v} . {v}_{c}}{α} ,$$

### Higher

$$\frac{{D}_{v} . {v}_{c}}{α}$$

corresponds to greater production increase and would be expected to shorten M_{RBC}, while lower

$$\frac{{D}_{v} . {v}_{c}}{α}$$

corresponds to greater clearance delay and would lengthen M_{RBC}. The model can be validated by comparing

$$\frac{{D}_{v} . {v}_{c}}{α}$$

to the change in M_{RBC} estimated from independent measurements of HbA1c and find the expected negative correlation (p<0.002), as shown in panel (F) of FIG. 5. Subjects whose modeled blood loss response shows transient (v, h) dynamics with relatively higher production increase have a greater reduction in M_{RBC}, as shown in panel (G) of FIG. 5.

The model thus finds that volume and hemoglobin dynamics of the typical RBC is significantly altered shortly after blood loss and remains altered for at least 21 days. Because P(v, h, t) is determined by these dynamics, the results imply that it should be possible to distinguish 21-day post-blood loss CBCs from pre-blood loss CBCs based only on P (v, h), without having to consider measurements of cell count or concentration like HGB, HCT, or reticulocyte count, among others. Machine learning methods were used to classify measurements of P(v, h) and achieved cross-validated performance of >98% (area under curve (AUC) 0.98) with multiple methods (e.g., quadratic discriminants, complex trees, etc.). By comparison, this classification by P(v, h) was significantly more accurate than classification using only the current gold standard count-based markers (e.g., HCT and reticulocyte count, accuracy 93%, AUC 0.90).

The single-cell model of routinely available clinical data described herein provides a mechanistic link between the (v, h) distribution and changes in the RBC age distribution. The model identifies delayed RBC clearance as an important unrecognized component of the compensatory response to blood loss, and it enables more nuanced and precise inferences about the homeostatic response to a fundamental pathologic process in different individuals.

Analysis begins with a mechanistic model and leads to identification of empirical changes in the (v, h) distribution that are associated with the response to blood loss. Importantly, the advantage of a mechanistic or semi-mechanistic modeling approach either in addition to or instead of a purely statistical or machine learning approach is that it provides a hypothesized physiologic context. Additional falsifiable predictions may then be deduced to provide further validation opportunities, as shown for instance in FIG. 6. A mechanistic model also enables assessment of counterfactuals, which is particularly important in the clinical context, where patient factors or pre-existing conditions not present in discovery or development cohorts might significantly compromise accuracy when inference methods are applied to real-world populations. An understanding of the mechanistic basis for an inference method of algorithm will increase the likelihood that these problematic situations can be anticipated and perhaps avoided. Such conditions may include transfusion, sickle cell disease, or mechanical RBC stresses altering RBC volume associated with disseminated intravascular coagulation, microangiopathic hemolytic anemia, and other pathologic processes.

The model can provide immediate clinical decision support by detecting RBC production or changes in RBC production earlier than the current gold standard reticulocyte count or other approaches used to infer production rate. In addition, the model can be used to quantify many aspects of human pathophysiologic state that cannot currently be measured at all or with desired accuracy. For example, the model can quantify RBC clearance rate at one point in time and changes in the clearance rate from one time to the next, such as in response to treatment or intervention or progression of disease or recovery. The model can also estimate the current age distribution of circulating RBCs, including the mean of the distribution and other statistics on that distribution (e.g., standard deviation, 95th percentile, etc.). In addition, the techniques described here can be expanded to compare the transient (v,h) dynamics in patients with active disease processes and determine which factors control the clearance/production ratio of a subject's blood loss response.

Two or more blood tests can be used to measure RBC dynamics with the model, with additional blood tests increasing the sensitivity of the measurements. For instance, all 7 daily CBCs for a patient who has been hospitalized for a week can be used to increase the sensitivity of the measurements. In an example, because the techniques described here combine measurements at multiple different timepoints, the previous measurements are stored and used to calibrate interpretation of the next measurement. More generally, each new measurement is combined with all previous measurements to update the average RBC dynamics over the entire period and also to estimate the most recent rates, where inference of the most recent rates is done using as many previous measurements as are available during a period of time equal to the RBC lifespan (e.g., about 100 days), and that duration of measurement inclusion will vary. In an example, all of the parameters are calibrated for analytic variation in different CBC and HbA1c measurement systems. Recalibration can be performed every so often by collecting CBC, HbA1c, and additional data, such as continuous glucose monitoring or RBC labeling data, to make sure the patient's estimated RBC production and clearance rates and age distributions are the same regardless of the measurements systems used.

In some examples, the model can be used to infer aspects of an individual's pathophysiologic state that are related to RBC dynamics, such as rate of change in white blood cell (WBC) count based on bone marrow co-regulation of the RBC population and detailed inference of RBC population dynamics with the method, rate of change in platelet (PLT) count based on bone marrow co-regulation of the RBC population and detailed inference of RBC population dynamics with the method, rate of blood loss, rate of overall bone marrow cellular output, such as following bone marrow transplant or gene therapy treatment, or combinations of them, among others.

The model is applicable to a wide range of diagnostic, treatment, and monitoring applications. For example, the model enables earlier and more accurate diagnosis of anemia by earlier and more sensitive detection of changes in RBC production rate than reticulocyte count, the current gold standard. The model also allows for earlier and more accurate diagnosis of anemia by earlier and more sensitive detection of changes in RBC clearance rate. There is currently no other way to estimate RBC clearance rate, and it is often modulated in anemia, for instance, in response to decreased RBC production. Similarly, the model enables early diagnosis of infection and malignancy (e.g., colon cancer, leukemia, other malignancy), among other pathological conditions, by detecting decreased RBC production or decreased RBC clearance, or both. The high sensitivity of the model can also allow it to distinguish autoimmunity, malignancy, infection, and other conditions by detecting and comparing subtle changes in RBC population dynamics.

In some examples, the model more accurately diagnoses diabetes by adjusting the HbA1c test for a patient's mean RBC age. In doing so, both the false positive rate of the HbA1c test (e.g., identifying individuals with high mean RBC age whose HbA1c is elevated because RBCs are old on average not because of hyperglycemia) and its false negative rate (e.g., identifying individuals whose HbA1c is normal despite hyperglycemia because RBCs are young on average) is reduced.

Estimates of RBC production rate, clearance rate, statistics on the RBC age distribution, and other measurements enabled by the model can also be used to select and monitor treatments for any of the conditions mentioned above. For instance, an iron supplementation dose administered to patients diagnosed with anemia can be reduced when RBC clearance rate is normal. As another example, empirical antibiotic selection for infectious disease can be confirmed by a finding of normalization of RBC production or clearance rates. Bone marrow engraftment following transplant for gene therapy can be monitored by estimating RBC production and clearance rates, and acuity of patient care can be informed. Diabetes monitoring with HbA1c can be personalized by adjusting frequent HbA1c measurements for the patient's RBC age and using the adjusted-HbA1c to guide decisions about treatment maintenance, reduction, or intensification. In general, any disease or treatment which is thought to alter net rates of change in RBC, WBC, or PLT populations can be more accurately monitoring by incorporating the techniques described here into current management protocols and algorithms.

In an example, a patient being screened for diabetes is found to have a HbA1c level of 5.5%. Ordinarily, the patient would be diagnosed as non-diabetic (e.g., because diabetic HbA1c for the patient is >6.5%). Using the techniques described here, the patient is found to have a short mean RBC age (e.g., less than about 45 days). This short mean RBC age means the patient's glycemia is about 135 mg/dL, a level diagnostic of diabetes which, for someone with a more typical mean RBC age 53 days, would correspond to an HbA1c of 6.6%. Using the personalized and more accurate assessment of glycemia enabled by the methods described here, patient treatment is initiated with lifestyle modification, consideration of metformin, and follow-up monitoring with continuous glucose monitoring, oral glucose tolerance, or combinations of them, among other approaches.

As another example, a patient being screened for diabetes has a HbA1c of 6.6% and would ordinarily be diagnosed with diabetes, but using the methods described here is shown to have a long mean RBC age of 60 days or greater. The long mean RBC age means that the patient's glycemia is actually below the diabetic range, and diabetic treatment is withheld as a result of this personalized and more accurate information.

As another example, a patient with diabetes is monitored for consideration of treatment intensification and is found to have an HbA1c of 6.9%, normally consistent with well-controlled glycemia and not indicating treatment intensification. Using the methods described here, the patient's mean RBC age is determined to be 45 days or less, meaning that an HbA1c of 6.9% corresponds to significant hyperglycemia. The patient's treatment regimen is intensified with addition of a second-line medication.

As another example, a patient with diabetes is monitored for consideration of treatment intensification and is found to have an HbA1c of 7.6%, normally consistent with insufficiently-controlled glycemia and indicating treatment intensification. Using the methods described here, the patient's mean RBC age is determined to be 60 days or higher, meaning that an HbA1c of 7.6% corresponds to well-controlled glycemia. The patient's treatment regimen is maintained at current levels.

As another example, a hospitalized patient is monitored for infection. The patient's white blood cell (WBC) count has increased from 5 to 6, but is still normal (<11), and there is no evidence of infection. Using the methods described here, the patient's

$$\frac{{D}_{v} . {v}_{c}}{α}$$

is found to decrease from 40 to 30, corresponding to a homeostatic response to infection. Blood, sputum, and urine culture tests are performed, and the patient is treated with empiric antibiotics.

As another example, a hospitalized patient appears stable, but

$$\frac{{D}_{v} . {v}_{c}}{α}$$

is found to decrease from 30 to 20, consistent with acute blood loss. Endoscopy and ultrasound studies are indicated to identify the source of any occult bleeding.

As another example, an outpatient at a healthy annual physical is found to have normal WBC, RBC, and platelet (PLT) counts, though all are in the lower half of their reference intervals. Nothing further would normally be done, but

$$\frac{{D}_{v} . {v}_{c}}{α}$$

is found to be less than 10. The patient is referred for bone marrow biopsy and imaging to search for primary tumors.

As another example, a cancer patient is being treated with one of many cancer treatments, for instance anthracyclines and taxanes, associated elevated risk for platelet suppression. The patient's

$$\frac{{D}_{v} . {v}_{c}}{α}$$

is 50, consistent with robust platelet production, and the drug dose is increased until the next monitoring visit.

The techniques described herein are typically practiced using peripheral blood samples obtained using known collection methodology that typically preserves RBCs intact (e.g., a blood draw with an appropriate amount of vacuum (draw) and a needle large enough to allow the RBCs to be collected without substantial hemolysis, e.g., a needle of at least 25 g or larger). The measurements are preferably made within 24, 12, or 6 hours of collection. Reticulocyte and CBC measurements can be made using any methods or devices known in the art that can measure both RBC volume (e.g., using low angle (2°-3°) scatter detection) and hemoglobin mass or concentration (e.g., using high angle (5°-15°) scatter detection).

The blood sample can be used with a hematology analyzer, an immunoassay analyzer, a hemoglobin testing system, and other appropriate blood testing apparatuses and devices. In some implementations, a hemanalyzer or hematology analyzer measures characteristics of the blood sample. The hemanalyzer can be, for example, a manual, semi-automated, or automated hematology analyzer. Hemanalyzers useful in the present methods can use any appropriate detection method known in the art, e.g., flow cytometry or optical or image-based analysis or impedance based. Hemanalyzers useful in the present methods and systems can measure the parameters of the CBC described herein, such as, for example, the RBC cell volume, and at least one of the cell hemoglobin concentration and cell hemoglobin mass.

All 28 subjects (18 male, 10 female) enrolled in the study described herein were healthy and athletically active individuals aged 18 to 40 on the day of enrollment. The study size provided at least 4 same-sex biological replicates and allowed for the possibility of a 50% dropout during the study. Subjects were excluded from enrollment if they participated in competitive sporting events during the study procedures, or if they were a member of a registered anti-doping testing pool for any international sporting federations, national anti-doping organizations, or professional sporting organizations.

Prior to each blood collection, subjects were seated with their feet on the floor for a minimum of ten minutes per World Anti-Doping Agency blood collection guidelines. After the ten-minute equilibration period, blood was collected via venipuncture of an antecubital vein into one 6 mL serum-separator tube and one 6 mL BD Vacutainer™ K₂EDTA tube (produced by Becton, Dickinson and Company, Franklin Lakes, N.J.). After collection, whole blood samples were immediately refrigerated until analysis. Additional aliquots were stored at −80 C for hemoglobin A1c (HbA1c) measurement.

Whole blood samples collected in K₂EDTA tubes were measured for a CBC plus reticulocyte percent using both a Sysmex XT-2000i (produced by Sysmex America, Inc., Lincolnshire, Ill.) and a Siemens Advia 2120i (produced by Siemens Medical Solutions USA, Inc., Malvern, Pa.). Briefly, samples were brought from refrigerated to room temperature while on a nutating mixer for at least 15 minutes prior to analysis. All samples were measured in duplicate on both instruments. All samples were collected in Salt Lake City, Utah, at either the Sports Medicine Research and Testing Laboratory (SMRTL) or the University of Utah Hospital. The approximate altitude at these locations is 1400 m above sea level. All subjects in the study were residents at this altitude and are assumed to be adapted to the environment.

CBCs were measured roughly every other day for a week for all subjects, and the model was used to infer each subject's baseline RBC population dynamics between these 4 timepoints (e.g., t=1, 3, 5, and 7 days). At t=1, b(v, h, t=1) is measured and is used to estimate source terms extending back in time by a number of days equivalent to the RBC lifespan (LS): b(v, h, (1−LS)<=t<1)=b(v, h, t=1). The RBC age distribution is assumed to be uniform with nominal LS=105 days (Cohen et al., 2008). The first CBC provides a sample of P(v, h, 1), and equation (1) is used to estimate the parameters characterizing the RBC population dynamics at baseline: p₁=(α₁, β_{v,1}, β_{h,1}, D_{v,1}, D_{h,1}, v_{c,1}) (Higgins and Mahadevan, 2010; Patel et al., 2015). The transient dynamics between t=1 and t=3 can be estimated using p₁ and equation (1). Initial conditions at t=1 are determined by integrating equation (1) for LS−2 days with a source term equal to b(v, h, 1). The CBC measured on day 3 (t=3) provides a direct estimate of b(v, h, 3) and a sample of P(v, h, 3). Equation (1) is then used to estimate p₃, the parameters characterizing the transient dynamics between t=1 and t=3. This process is repeated for each successive CBC to provide quantification of the transient dynamics (e.g., the quantified dynamics shown in FIG. 3B).

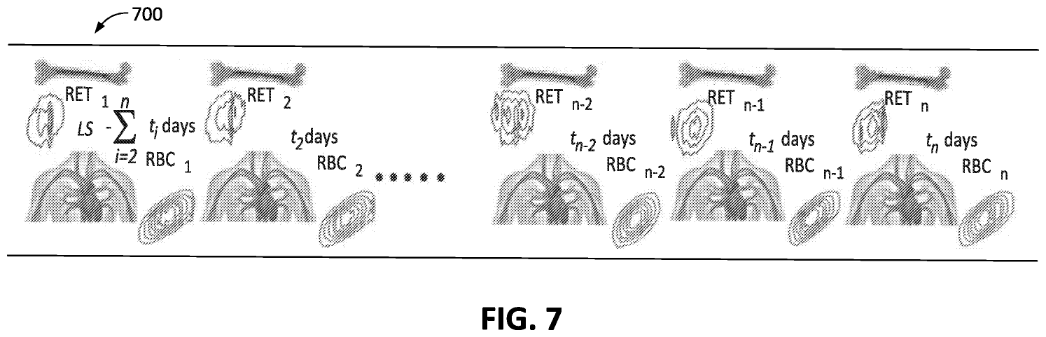

FIG. 7 is a schematic illustration of the process of modeling integrated serial CBCs into the parameter estimation process in a piecewise manner. The first CBC (represented at portion 700 of FIG. 7) is assumed to be at steady state, and the model (e.g., as defined in equation (1)) is used to estimate dynamic parameters which produce RBC₁ given RET₁. In some examples, v and h are normalized by their sample population means. These model parameters and RET₁ are then used to estimate the initial condition leading to timepoint t₂, and the model estimates the dynamics between timepoints t₁ and t₂. These steps for timepoint t₂ are then repeated to estimate the transient dynamics between each successive timepoint.

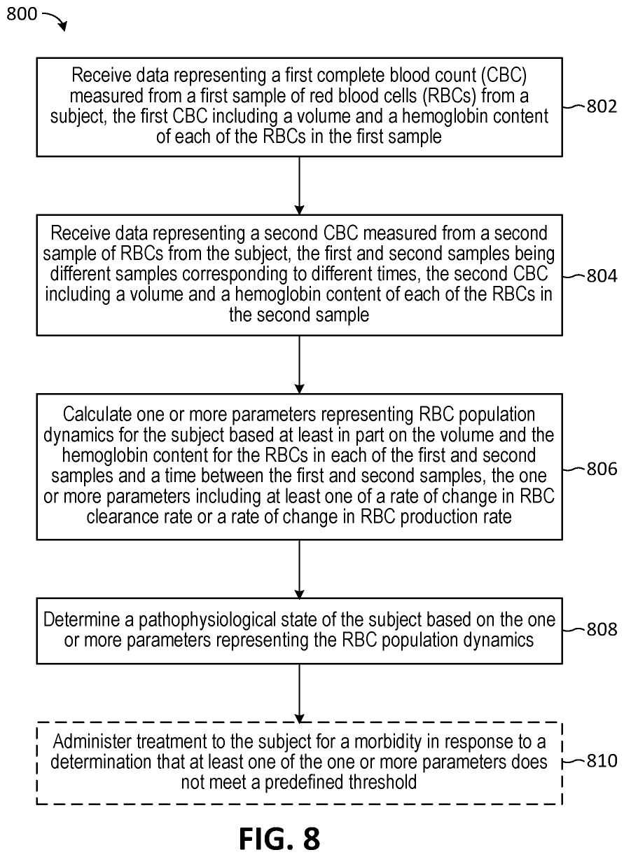

FIG. 8 illustrates a process 800 for determining a pathophysiological state of a patient in accordance with the techniques described herein. The process 800 can be implemented by, for example, by a computing device (such as the computing device 900 shown in FIG. 9) configured to carry out operations to implement the model and other techniques described here and with reference to FIGS. 1-7.

Operations of the process 800 include receiving (802) data representing a first complete blood count (CBC) measured from a first sample of red blood cells (RBCs) from a subject, the first CBC including a volume and a hemoglobin content of each of the RBCs in the first sample. Data representing a second CBC measured from a second sample of RBCs from the subject is also received (804), the second CBC including a volume and a hemoglobin content of each of the RBCs in the second sample. The first and second samples are different samples corresponding to different times. In an example, each of the first and second CBCs are measured by a hematology analyzer. The process can be generalized to any number of additional blood tests, with additional blood tests increasing the sensitivity of the process. For instance, all 7 daily CBCs for a patient who has been hospitalized for a week can be used to increase the sensitivity of the method.

One or more parameters representing RBC population dynamics for the subject are calculated (806) based at least in part on the volume and the hemoglobin content for the RBCs in each of the first and second samples and a time between the first and second samples. The one or more parameters include at least one of a rate of change in RBC clearance rate or a rate of change in RBC production rate. In an example, the one or more parameters include at least one of a rate of RBC volume change or a variation of RBC volume change. In an example, the one or more parameters include at least one of a rate of RBC hemoglobin reduction or a variation of RBC hemoglobin reduction. In an example, calculating the one or more parameters representing RBC population dynamics for the subject includes calculating a probability density of RBC volume, RBC hemoglobin content, and RBC age as a function of time. The probability density as a function of time can be determined according to the expression −∇(Pf)+∇·(D∇P)+b(v, h)−d(v, h), where P is a RBC volume-hemoglobin probability distribution, f is a drift term, D is a diffusion matrix, b(v, h) is RBC production defined as a function of RBC volume and hemoglobin content, and d(v, h) is RBC clearance defined as a function of RBC volume and hemoglobin content.

A pathophysiological state of the subject id determined (808) based on the one or more parameters representing the RBC population dynamics. In an example, determining the pathophysiological state of the subject includes determining, based on the one or more parameters, at least one of a RBC clearance rate, a RBC production rate, or a RBC age distribution. In an example, determining the pathophysiological state of the subject includes determining, based on the one or more parameters, at least one of a rate of change in white blood cell count, a rate of change in platelet count, a rate of blood loss, or a rate of bone marrow cellular output. In an example, determining the pathophysiological state of the subject includes determining, based on the one or more parameters, information indicative of a degree of morbidity of the subject, the information including at least one of: information indicative of the presence of an infection, information indicative of the presence of malignancy, information indicative of the presence of anemia, or information indicative of the presence of diabetes. In an example, a RBC age distribution for the subject is calculated based on the one or more parameters and at least one of the first CBC or the second CBC.

In an example, the process 800 further includes administering (810) treatment to the subject for a morbidity in response to a determination that at least one of the one or more parameters does not meet a predefined threshold. The optional nature of this step is indicated by the dash outlined in FIG. 8. For instance, in an example, a hemoglobin A1c (HbA1c) measurement for the subject is received, a HbA1c level indicative of diabetes or prediabetes for the subject is determined by adjusting a nominal HbA1c level based on the RBC age distribution, and a treatment for diabetes or prediabetes is administered to the subject in response to a determination that the HbA1c measurement for the subject exceeds the HbA1c level for the subject. In an example, a HbA1c measurement for the subject is received, a HbA1c level indicative of diabetes or prediabetes for the subject is determined by adjusting a nominal HbA1c level based on the RBC age distribution, and treatment for diabetes or prediabetes to the subject is adjusted in response to a determination that the HbA1c measurement for the subject exceeds the HbA1c level for the subject. In an example, a dose of iron supplementation is administered to the subject in response to a determination that the change in the RBC clearance rate does not meet a predefined threshold.

FIG. 9 is a block diagram of an example computing system 900 that can be used to carry out any of the techniques described herein. The computing system 900 can receive data from a user or from external measurement and testing systems (e.g., a hematology analyzer, a hemoglobin testing system, an immunoassay analyzer, or combinations of them, among others), store the data, and process the data. The computing system 900 can further make determinations about a diagnosis or treatment plan for a subject associated with the data, or can present data to a healthcare professional to aid the healthcare professional in making such a determination. The system 900 includes a processor 910, a memory 920, a storage device 930, and one or more input/output interface devices 940. Each of the components 910, 920, 930, and 940 can be interconnected, for example, using a system bus 950.

The processor 910 is capable of processing instructions for execution within the system 900. The term “execution” as used here refers to a technique in which program code causes a processor to carry out one or more processor instructions. In some implementations, the processor 910 is a single-threaded processor. In some implementations, the processor 910 is a multi-threaded processor. The processor 910 is capable of processing instructions stored in the memory 920 or on the storage device 930. The processor 910 may execute operations such as those described with reference to the process 800 of FIG. 8.

The memory 920 stores information within the system 900. In some implementations, the memory 920 is a computer-readable medium. In some implementations, the memory 920 is a volatile memory unit. In some implementations, the memory 920 is a non-volatile memory unit.

The storage device 930 is capable of providing mass storage for the system 900. In some implementations, the storage device 930 is a non-transitory computer-readable medium. In various different implementations, the storage device 930 can include, for example, a hard disk device, an optical disk device, a solid-state drive, a flash drive, magnetic tape, or some other large capacity storage device. In some implementations, the storage device 930 may be a cloud storage device, e.g., a logical storage device including one or more physical storage devices distributed on a network and accessed using a network. In some examples, the storage device may store long-term data, such as data related to CBC measurements, RBC population dynamics, diagnostic or treatment thresholds, or combinations of them, among other data.

The input/output interface devices 940 provide input/output operations for the system 900. In some implementations, the input/output interface devices 940 can include one or more of a network interface devices, e.g., an Ethernet interface, a serial communication device, e.g., an RS-232 interface, and/or a wireless interface device, e.g., an 802.11 interface, a 3G wireless modem, a 4G wireless modem, etc. A network interface device allows the system 900 to communicate, for example, transmit and receive data. In some implementations, the input/output device can include driver devices configured to receive input data and send output data to other input/output devices, e.g., keyboard, printer and display devices 960. In some implementations, mobile computing devices, mobile communication devices, and other devices can be used.

Referring to FIG. 8, the process 800 can be realized by instructions that upon execution cause one or more processing devices to carry out the processes and functions described above, for example, to determine a pathophysiological state of a patient in accordance with the techniques described herein. Such instructions can include, for example, interpreted instructions such as script instructions, or executable code, or other instructions stored in a computer readable medium.

The system 900 can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

In some examples, the system 900 is contained within a single integrated circuit package. A system 900 of this kind, in which both a processor 910 and one or more other components are contained within a single integrated circuit package and/or fabricated as a single integrated circuit, is sometimes called a microcontroller. In some implementations, the integrated circuit package includes pins that correspond to input/output ports, e.g., that can be used to communicate signals to and from one or more of the input/output interface devices 940.

Although an example processing system has been described in FIG. 9, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification, such as storing, maintaining, and displaying artifacts can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, or a combination of one or more of them.

The term “system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM, DVD-ROM, and Blu-Ray disks. The processor and the memory can be supplemented by, or incorporated in, special purpose logic circuitry. Sometimes a server is a general purpose computer, and sometimes it is a custom-tailored special purpose electronic device, and sometimes it is a combination of these things. Implementations can include a back end component, e.g., a data server, or a middleware component, e.g., an application server, or a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

The following publications are incorporated by reference in their entirety.
1. Baron C S, Kester L, Klaus A, Boisset J-C, Thambyrajah R, Yvernogeau L, Kouskoff V, Lacaud G, van Oudenaarden A, Robin C. 2018. Single-cell transcriptomics reveal the dynamic of haematopoietic stem cell production in the aorta. Nat Commun 9:2517. doi:10.1038/s41467-018-04893-3.
2. Beutler E, Waalen J. 2006. The definition of anemia: what &lt;em&gt;is&lt;/em&gt; the lower limit of normal of the blood hemoglobin concentration? Blood 107:1747 LP-1750.
3. Bosman G J C G M, Werre J M, Willekens F L A, Novotny V M J. 2008. Erythrocyte ageing in vivo and in vitro: structural aspects and implications for transfusion. Transfus Med 18:335-347. doi:10.1111/j.1365-3148.2008.00892.x
4. Bunn H F. 2013. Erythropoietin. Cold Spring Harb Perspect Med 3:a011619. doi:10.1101/cshperspect.a011619
5. Bunn H F, Haney D N, Kamin S, Gabbay K H, Gallop P M. 1976. The biosynthesis of human hemoglobin A1c. Slow glycosylation of hemoglobin in vivo. J Clin Invest 57:1652-1659. doi:10.1172/JCI108436
6. Chaudhury A, Noiret L, Higgins J M. 2017. White blood cell population dynamics for risk stratification of acute coronary syndrome. Proc Natl Acad Sci 114:12344-12349.
7. Cohen R M, Franco R S, Khera P K, Smith E P, Lindsell C J, Ciraolo P J, Palascak M B, Joiner C H. 2008. Red cell life span heterogeneity in hematologically normal people is sufficient to alter HbA1c. Blood 112:4284-4291. doi:10.1182/blood-2008-04-154112.
8. d'Onofrio G, Chirillo R, Zini G, Caenaro G, Tommasi M, Micciulli G. 1995. Simultaneous measurement of reticulocyte and red blood cell indices in healthy subjects and patients with microcytic and macrocytic anemia. Blood.
9. Dijkstra A, Lenters-Westra E, De Kort W, Bokhorst A G, Bilo H J G, Slingerland R J, Vos M J. 2017. Whole blood donation affects the interpretation of hemoglobin A1c. PLoS One. doi:10.1371/journal.pone.0170802
10. Dornhorst A C. 1951. Analytical Review: The Interpretation of Red Cell Survival Curves. Blood 6:1284 LP-1292.
11. Franco R S. 2008. The measurement and importance of red cell survival. Am J Hematol 84:109-114. doi:10.1002/ajh.21298.
12. Franco R S, Puchulu-Campanella M E, Barber L A, Palascak M B, Joiner C H, Low P S, Cohen R M. 2013. Changes in the properties of normal human red blood cells during in vivo aging. Am J Hematol. doi:10.1002/ajh.23344.
13. Gifford S C, Derganc J, Shevkoplyas S S, Yoshida T, Bitensky M W. 2006. A detailed study of time-dependent changes in human red blood cells: from reticulocyte maturation to erythrocyte senescence. Br J Haematol 135:395-404. doi:10.1111/j.1365-2141.2006.06279.x
14. Giustacchini A, Thongjuea S, Barkas N, Woll P S, Povinelli B J, Booth C A G, Sopp P, Norfo R, Rodriguez-Meira A, Ashley N, Jamieson L, Vyas P, Anderson K, Segerstolpe Å, Qian H, Olsson-Stromberg U, Mustjoki S, Sandberg R, Jacobsen S E W, Mead A J. 2017. Single-cell transcriptomics uncovers distinct molecular signatures of stem cells in chronic myeloid leukemia. Nat Med 23:692.
15. Higgins J M, Mahadevan L. 2010. Physiological and pathological population dynamics of circulating human red blood cells. Proc Natl Acad Sci 107:20587-20592. doi:10.1073/pnas.1012747107.
16. Jelkmann W, Lundby C. 2011. Blood doping and its detection. Blood 118:2395 LP-2404.
17. Khera P K, Lindsell C J, Joiner C H, Cohen R M, Franco R S. 2013a. Measurement Of Erythrocyte Survival &lt;em&gt;In Vivo&lt;/em&gt; using a Stable Isotope Label In Sickle Cell Anemia. Blood 122:2223 LP-2223.
18. Khera P K, Lindsell C J, Joiner C H, Cohen R M, Franco R S. 2013b. Measurement Of Erythrocyte Survival In Vivo using a Stable Isotope Label In Sickle Cell Anemia. Blood 122:2223 LP-2223.
19. Kim Y R, Ornstein L. 1983. Isovolumetric sphering of erythrocytes for more accurate and precise cell volume measurement by flow cytometry. Cytometry 3:419-427. doi:10.1002/cyto.990030606.
20. Lew V L, Raftos J E, Sorette M, Bookchin R M, Mohandas N. 1995. Generation of normal human red cell volume, hemoglobin content, and membrane area distributions by &quot;birth&quot; or regulation? Blood 86:334 LP-341.
21. Malka R, Delgado F F, Manalis S R, Higgins J M. 2014. In Vivo Volume and Hemoglobin Dynamics of Human Red Blood Cells. PLOS Comput Biol 10:e1003839.
22. Malka R, Nathan D M, Higgins J M. 2016. Mechanistic modeling of hemoglobin glycation and red blood cell kinetics enables personalized diabetes monitoring. Sci Transl Med 8:359ra130 LP-359ra130. doi:10.1126/scitranslmed.aaf9304.
23. Mohandas N, Kim Y R, Tycko D H, Orlik J, Wyatt J, Groner W. 1986. Accurate and independent measurement of volume and hemoglobin concentration of individual red cells by laser light scattering. Blood 68:506 LP-513.
24. Patel H H, Patel H R, Higgins J M. 2015. Modulation of red blood cell population dynamics is a fundamental homeostatic response to disease. Am J Hematol. doi:10.1002/ajh.23982.
25. Piva E, Brugnara C, Spolaore F, Plebani M. 2015. Clinical Utility of Reticulocyte Parameters. Clin Lab Med 35:133-163. doi:https://doi.org/10.1016/j.cll.2014.10.004.
26. Shalek A K, Satija R, Adiconis X, Gertner R S, Gaublomme J T, Raychowdhury R, Schwartz S, Yosef N, Malboeuf C, Lu D, Trombetta J J, Gennert D, Gnirke A, Goren A, Hacohen N, Levin J Z, Park H, Regev A. 2013. Single-cell transcriptomics reveals bimodality in expression and splicing in immune cells. Nature 498:236.
27. Shrestha R P, Horowitz J, Hollot C V., Germain M J, Widness J A, Mock D M, Veng-Pedersen P, Chait Y. 2016. Models for the red blood cell lifespan. J Pharmacokinet Pharmacodyn. doi:10.1007/s10928-016-9470-4.
28. Sieff C A. 2017. Regulation of erythropoiesis. uptodate.com.
29. Tusi B K, Wolock S L, Weinreb C, Hwang Y, Hidalgo D, Zilionis R, Waisman A, Huh J R, Klein A M, Socolovsky M. 2018. Population snapshots predict early haematopoietic and erythroid hierarchies. Nature 555:54.
30. Van Kampen N G. 2007. Stochastic Processes in Physics and Chemistry (Third Edition), North-Holland Personal Library. Amsterdam: Elsevier. doi:http://dx.doi.org/10.1016/B978-044452965-7/50011-8.
31. Waugh R E, Narla M, Jackson C W, Mueller T J, Suzuki T, Dale G L. 1992. Rheologic properties of senescent erythrocytes: loss of surface area and volume with red blood cell age. Blood 79:1351 LP-1358.
32. Willekens F L A, Werre J M, Groenen-Döpp YAM, Roerdinkholder-Stoelwinder B, De Pauw B, Bosman G J C G M. 2008. Erythrocyte vesiculation: a self-protective mechanism? Br J Haematol 141:549-556. doi:10.1111/j.1365-2141.2008.07055.x.

1. A method, comprising:
receiving data representing a first complete blood count (CBC) measured from a first sample of red blood cells (RBCs) from a subject, the first CBC including a volume and a hemoglobin content of each of the RBCs in the first sample;
receiving data representing a second CBC measured from a second sample of RBCs from the subject, the first and second samples being different samples corresponding to different times, the second CBC including a volume and a hemoglobin content of each of the RBCs in the second sample;
calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate; and
determining a pathophysiological state of the subject based on the one or more parameters representing the RBC population dynamics.

2. The method of claim 1, wherein determining the pathophysiological state of the subject includes determining, based on the one or more parameters, at least one of a RBC clearance rate, a RBC production rate, or a RBC age distribution.

3. The method of claim 1, wherein determining the pathophysiological state of the subject includes determining, based on the one or more parameters, at least one of a rate of change in white blood cell count, a rate of change in platelet count, a rate of blood loss, or a rate of bone marrow cellular output.

4. The method of claim 1, wherein determining the pathophysiological state of the subject includes determining, based on the one or more parameters, information indicative of a degree of morbidity of the subject, the information including at least one of: information indicative of the presence of an infection, information indicative of the presence of malignancy, information indicative of the presence of anemia, or information indicative of the presence of diabetes.

5. The method of claim 1, wherein the one or more parameters include at least one of a rate of RBC volume change or a variation of RBC volume change.

6. The method of claim 1, wherein the one or more parameters include at least one of a rate of RBC hemoglobin reduction or a variation of RBC hemoglobin reduction.

7. The method of claim 1, wherein calculating one or more parameters representing RBC population dynamics for the subject includes calculating a probability density of RBC volume, RBC hemoglobin content, and RBC age as a function of time.

8. The method of claim 7, wherein the probability density as a function of time is determined according to the expression −∇·(Pf)+∇·(D∇P)+b(v,h)−d(v,h), where P is a RBC volume-hemoglobin probability distribution, f is a drift term, D is a diffusion matrix, b(v,h) is RBC production defined as a function of RBC volume and hemoglobin content, and d(v,h) is RBC clearance defined as a function of RBC volume and hemoglobin content.

9. The method of claim 1, comprising calculating a RBC age distribution for the subject based on the one or more parameters and at least one of the first CBC or the second CBC.

10. The method of claim 9, comprising:
receiving a hemoglobin A1c (HbA1c) measurement for the subject;
determining a HbA1c level indicative of diabetes or prediabetes for the subject by adjusting a nominal HbA1c level based on the RBC age distribution; and
administering treatment for diabetes or prediabetes to the subject in response to a determination that the HbA1c measurement for the subject exceeds the HbA1c level for the subject.

11. The method of claim 9, comprising:
receiving a hemoglobin A1c (HbA1c) measurement for the subject;
determining a HbA1c level indicative of diabetes or prediabetes for the subject by adjusting a nominal HbA1c level based on the RBC age distribution; and
adjusting treatment for diabetes or prediabetes to the subject in response to a determination that the HbA1c measurement for the subject exceeds the HbA1c level for the subject.

12. The method of claim 1, comprising
administering a dose of iron supplementation to the subject in response to a determination that the change in the RBC clearance rate does not meet a predefined threshold.

13. A system, comprising:
one or more processors; and
memory storing instructions which, when executed by the one or more processors, cause the one or more processors to:
receive data representing a first complete blood count (CBC) measured from a first sample of red blood cells (RBCs) from a subject, the first CBC including a volume and a hemoglobin content of each of the RBCs in the first sample;
receive data representing a second CBC measured from a second sample of RBCs from the subject, the first and second samples being different samples corresponding to different times, the second CBC including a volume and a hemoglobin content of each of the RBCs in the second sample;
calculate one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate; and
determine a pathophysiological state of the subject based on the one or more parameters representing the RBC population dynamics.

14. A non-transitory computer-readable storage medium storing instructions which, when executed by one or more processors, cause the one or more processors to perform operations comprising:
receiving data representing a first complete blood count (CBC) measured from a first sample of red blood cells (RBCs) from a subject, the first CBC including a volume and a hemoglobin content of each of the RBCs in the first sample;
receiving data representing a second CBC measured from a second sample of RBCs from the subject, the first and second samples being different samples corresponding to different times, the second CBC including a volume and a hemoglobin content of each of the RBCs in the second sample;
calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate; and
determining a pathophysiological state of the subject based on the one or more parameters representing the RBC population dynamics.

15. A method, comprising:
receiving data representing a first complete blood count (CBC) measured from a first sample of red blood cells (RBCs) from a subject, the first CBC including a volume and a hemoglobin content of each of the RBCs in the first sample;
receiving data representing a second CBC measured from a second sample of RBCs from the subject, the first and second samples being different samples corresponding to different times, the second CBC including a volume and a hemoglobin content of each of the RBCs in the second sample;
calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate; and
administering treatment to the subject for a morbidity in response to a determination that at least one of the one or more parameters does not meet a predefined threshold.

16. The method of claim 15, wherein the morbidity includes at least one of an infection, a malignancy, anemia, or diabetes.

17. The method of claim 15, wherein the one or more parameters include at least one of a rate of RBC volume change or a variation of RBC volume change.

18. The method of claim 15, wherein the one or more parameters include at least one of a rate of RBC hemoglobin reduction or a variation of RBC hemoglobin reduction.

19. The method of claim 1, wherein calculating one or more parameters representing RBC population dynamics for the subject includes calculating a probability density of RBC volume, RBC hemoglobin content, and RBC age as a function of time.

20. The method of claim 19, wherein the probability density as a function of time is determined according to the equation −∇·(Pf)+∇·(D∇P)+b(v,h)−d(v,h), where P is a RBC volume-hemoglobin probability distribution, f is a drift term, D is a diffusion matrix, b(v,h) is RBC production defined as a function of RBC volume and hemoglobin content, and d(v,h) is RBC clearance defined as a function of RBC volume and hemoglobin content.

21. The method of claim 15, comprising
calculating a RBC age distribution for the subject based on the one or more parameters and at least one of the first CBC or the second CBC.

22. The method of claim 21, comprising:
receiving a hemoglobin A1c (HbA1c) measurement for the subject;
determining a HbA1c level indicative of diabetes or prediabetes for the subject by adjusting a nominal HbA1c level based on the RBC age distribution; and
administering treatment for diabetes or prediabetes to the subject in response to a determination that the HbA1c measurement for the subject exceeds the HbA1c level for the subject.

23. The method of claim 21, comprising:
receiving a hemoglobin A1c (HbA1c) measurement for the subject;
determining a HbA1c level indicative of diabetes or prediabetes for the subject by adjusting a nominal HbA1c level based on the RBC age distribution; and
adjusting treatment for diabetes or prediabetes to the subject in response to a determination that the HbA1c measurement for the subject exceeds the HbA1c level for the subject.

24. The method of claim 15, comprising
administering a dose of iron supplementation to the subject in response to a determination that the change in the RBC clearance rate does not meet a predefined threshold.

25. A system, comprising:
one or more processors; and
memory storing instructions which, when executed by the one or more processors, cause the one or more processors to:
receive data representing a first complete blood count (CBC) measured from a first sample of red blood cells (RBCs) from a subject, the first CBC including a volume and a hemoglobin content of each of the RBCs in the first sample;
receive data representing a second CBC measured from a second sample of RBCs from the subject, the first and second samples being different samples corresponding to different times, the second CBC including a volume and a hemoglobin content of each of the RBCs in the second sample;
calculate one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate; and
indicate treatment for the subject for a morbidity in response to a determination that at least one of the one or more parameters does not meet a predefined threshold.

26. A non-transitory computer-readable storage medium storing instructions which, when executed by one or more processors, cause the one or more processors to perform operations comprising:
receiving data representing a first complete blood count (CBC) measured from a first sample of red blood cells (RBCs) from a subject, the first CBC including a volume and a hemoglobin content of each of the RBCs in the first sample;
receiving data representing a second CBC measured from a second sample of RBCs from the subject, the first and second samples being different samples corresponding to different times, the second CBC including a volume and a hemoglobin content of each of the RBCs in the second sample;
calculating one or more parameters representing RBC population dynamics for the subject based at least in part on the volume and the hemoglobin content for the RBCs in each of the first and second samples and a time between the first and second samples, the one or more parameters including at least one of a rate of change in RBC clearance rate or a rate of change in RBC production rate; and
indicating treatment for the subject for a morbidity in response to a determination that at least one of the one or more parameters does not meet a predefined threshold.
